(19) United States
(12) Patent Application Publication
(10) Pub. No.: US 2017/0007276 A1
(43) Pub. Date: Jan. 12, 2017

(54) GALLSTONE REMOVAL THROUGH CHOLECYSTODUODENAL FISTULA BY ANASTOMOSIS DEVICE

(71) Applicant: Empire Technology Development LLC, Wilmington, DE (US)

(72) Inventors: Debasish PRADHAN, Sambalpur (IN); Nikhil Ramchandra KATRE, Thane (IN); Salman KAPADIA, Seoni (IN)

(21) Appl. No.: 14/924,341

(22) Filed: Oct. 27, 2015

(30) Foreign Application Priority Data
Jul. 7, 2015 (IN) 2058/DEL/2015

(51) Int. Cl. A61B 17/22; A61B 17/11; A61B 18/20; A61B 18/14; A61B 17/32; A61B 17/3211

(52) U.S. Cl. CPC A61B 17/22; A61B 17/320068; A61B 17/3211; A61B 18/20; A61B 18/1492; A61B 17/1114; A61B 2017/1139

(57) ABSTRACT

An anastomosis device may include a base member defining a passageway between a first end and a second end of the base member, a first support member connected to the base member proximal to the first end, and a second support member connected to the base member proximal to the second end. The base member may have a cutting element to cut tissue from the first end. At least one of the first support member and the second support member may be movable along a length of the base member to secure a portion of tissue between the first and second support members.

### BACKGROUND

The gallbladder is a small, pear-shaped pouch below the liver in the upper right portion of the abdomen of a human body. The gallbladder connects to the liver and the bowel through the bile duct, which is also attached the pancreas. The gallbladder may store bile produced by the liver until needed. Bile may be used to help digest fats or other substances. As food is ingested, the gallbladder contracts to push and release bile through ducts and into the small intestine to help with digestion. The ducts include hepatic ducts (which carry bile out of the liver), cystic ducts (which take bile to and from the gallbladder), and the common bile duct (which takes bile from the cystic and hepatic ducts to the small intestine).

However, due to a variety of reasons, gallstones may develop within the gallbladder or bile ducts. Gallstones may form when the bile hardens into pieces of stone-like material. The gallstones may cause severe pain in the upper abdomen. Gallstones can block the normal flow of bile if the gallstones move from the gallbladder and lodge in any of the ducts carrying bile from the liver to the small intestine.

There are several different types of gallstones. For example, cholesterol gallstones, a common type of gallstone, may form as a result of excess cholesterol in the bile and are common in overweight patients and patients with ileal disease or with small bowel resection. Pigment gallstones, a relatively less common type of gallstone, may form when there is excess bilirubin in the bile. Pigment gallstones may also develop as a result of liver disease, hemolytic anemias, cirrhosis, an infection in the bile duct, or an inherited blood disorder, such as sickle cell anemia, where blood cells are broken down by the body too quickly.

About 10% of the general population has gallstones. Gallstones are approximately two times more common in females than in males. Overweight women in the middle years may, in particular, be susceptible to gallstones. Approximately 5-20% of women between 20-55 years and 25-30% of women over 55 years have gallstones. The prevalence for males is approximately half than that of women in any given age group.

Gallstones may cause sudden or long-term inflammation to the gallbladder (e.g., acute or chronic cholecystitis), inflammation of the pancreas and bile ducts, gallstone ileus, jaundice, and/or gallbladder cancer. Untreated gallstones may be life-threatening.

Surgery may be used to remove the gallbladder (i.e. cholecystectomy) in order to treat or remove the gallstones. About 500,000 Americans undergo cholecystectomy every year and this number has been increasing. Surgery may result in many different complications, such as injury to one of the bile ducts. If a bile duct is torn, bile can leak into the abdominal cavity, which may result in a painful and potentially life-threatening infection, as well as an additional surgery.

Additionally, 10-15% of patients suffer from post-cholecystectomy syndrome as a result of the cholecystectomy. This syndrome is characterized by a continuation of symptoms attributed to gallbladder disease or the development of new gastrointestinal symptoms.

Aside from the complications, surgery is also not ideal since there are benefits to keeping the gallbladder and not all patients may be able to have the surgery. Other techniques to remove the gallstones may not prevent gallstone reoccurrence and require complex procedures.

### SUMMARY

According to one embodiment, an anastomosis device may include a base member defining a passageway between a first end and a second end of the base member, a first support member connected to the base member proximal to the first end, and a second support member connected to the base member proximal to the second end. The base member may have a cutting element to cut tissue from the first end. At least one of the first support member and the second support member may be movable along a length of the base member to secure a portion of tissue between the first and second support members.

According to another embodiment, an anastomosis method may include advancing an anastomosis device into a first cavity with a first tissue wall, cutting the first tissue wall with the first end of the anastomosis device, advancing the first support member through the first tissue wall to a second tissue wall of a second cavity, cutting the second tissue wall with the first end of the anastomosis device, advancing the first support member through the second tissue wall, and clamping the first and second tissue walls between the first support member and the second support member. The anastomosis device may include a base member defining an inner passage between a first end and a second end, and a first support member and a second support member extending outwardly from the base member.

According to yet another embodiment, a method for accessing a gallbladder may include cauterizing a duodenum tissue wall of a duodenum with a first end of an anastomosis device, advancing the anastomosis device at least partially through the duodenum tissue wall, and cauterizing a gallbladder tissue wall of the gallbladder with the first end of the anastomosis device. A base member of the anastomosis device may define a passageway through the anastomosis device from the first end to the second end of the anastomosis device. The method may further include advancing the anastomosis device at least partially through the gallbladder tissue wall and clamping the duodenum tissue wall to the gallbladder tissue wall around an outer circumference of the base member between the first end and a second end. The anastomosis device may at least partially remain in the duodenum.

According to still another embodiment, a method for cleaning a cavity within a body may include advancing an endoscope to a tissue wall of a first cavity within the body, advancing a needle through the endoscopic port and the tissue wall into a second cavity, rinsing the second cavity with the needle, and retracting the needle into the endoscopic port. Adistal end of the endoscope may include an endoscopic port,

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIGS. 1A-1B are cross-sectional, side views of an anastomosis device in a undeployed position and a deployed position, respectively, according to one embodiment.

FIGS. 2A-2B are side views of an anastomosis device in a undeployed position and a deployed position, respectively, and attached to a delivery device according to another embodiment.

FIGS. 3A-3F are side views of an anastomosis device moving through two tissue walls and from a undeployed position to a deployed position according to one embodiment.

FIG. 3G is a cross-sectional, side view of the anastomosis device of FIG. 3F.

FIG. 3H is a cross-sectional, side view of the anastomosis device of FIG. 3F without tissue walls.

FIGS. 4A-4B are side views of an anastomosis device with a balloon in an inflated state and a deflated state, respectively, according to one embodiment.

FIG. 5A is a side view of an anastomosis device attached to a delivery device according to another embodiment.

FIGS. 5B-5C are perspective views of the anastomosis device of FIG. 5A with a sheath in an unattached position and an attached position, respectively.

FIG. 6A is a partially transparent, side view of an anastomosis device according to one embodiment in the duodenum.

FIG. 6B is a partially transparent, side, enlarged view of the anastomosis device of FIG. 6A.

FIG. 6C is a partially transparent, side view of a needle extending into the gallbladder from the anastomosis device of FIG. 6A.

FIG. 6D is a partially transparent, side view of the anastomosis device of FIG. 6A within the duodenum.

FIG. 6E is a partially transparent, side view of the anastomosis device of FIG. 6A extending partially out of the duodenum.

FIG. 6F is a partially transparent, partially cross-sectional, side view of the anastomosis device of FIG. 6A extending into the gallbladder from the duodenum.

FIG. 6G is a partially transparent, side view of the anastomosis device of FIG. 6A anastomosing the duodenum and the gallbladder.

FIG. 6H is a partially transparent, partially cross-sectional, side view of the anastomosis device of FIG. 6A draining the gallbladder into the duodenum.

FIG. 7 is a flow diagram of an anastomosis method according to one embodiment.

FIG. 8 is a flow diagram of a method for accessing a gallbladder according to another embodiment.

FIG. 9 is a flow diagram of a method for cleaning a cavity within a body according to yet another embodiment.

### DETAILED DESCRIPTION

In the following detailed description, reference is made to the accompanying drawings, which form a part thereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Referring generally to the disclosure, described herein is an anastomosis device and methods to provide access to the gallbladder through the intestinal tract and allow gallstones to be easy and conveniently removed. More specifically, the anastomosis device may create a passage between the duodenum and the gallbladder to provide access to the gallbladder. The gallstones may be removed from the gallbladder through the anastomosis device. The gallstones may be moved into the duodenum to be excreted from the body or removed from the body through a delivery device.

Generally speaking, the anastomosis device is delivered into the body of the patient through, for example, the mouth and may follow the digestive track to advance into the duodenum. The anastomosis device may first clean the gallbladder by extending a needle into the gallbladder from the duodenum. Once the gallbladder has been cleaned with a needle, the anastomosis device may begin to join the duodenum and the gallbladder. For example, a first end of the anastomosis device may cauterize the duodenum tissue wall to create an aperture in the duodenum tissue wall. Part of the anastomosis device may be advanced through the cauterized aperture and may be extended toward the gallbladder tissue wall. Subsequently, the anastomosis device may cauterize the gallbladder tissue wall to create an aperture in the gallbladder tissue wall and the anastomosis device may be advanced within the gallbladder. One of the support members of the anastomosis device may remain in the duodenum while the other support member may advance into the gallbladder. Accordingly, once the anastomosis device is in position, at least one of the support members may move toward the other to pinch the tissue walls between the support members and secure the anastomosis device to the tissue. The anastomosis device may include an inner passageway to allow material to move between the gallbladder and the duodenum. The gallbladder may be further cleaned with the delivery device and/or the delivery device may be detached from the anastomosis device, allowing the anastomosis device to continue draining the gallbladder into the duodenum.

Accordingly, with the anastomosis device and methods described further herein, bodily material, such as gallstones, may be removed from the body non-invasively and the inside of certain organs, such as the gallbladder, may be easily accessed. Although medicine may be used with the anastomosis device to capture and break the gallstones, the anastomosis device may allow the gallstones to be easily treated and completely extracted mechanically. Further, the anastomosis device may prevent a gap between the duodenum and the gallbladder after attachment, which prevents any leakage of bile to prevent inflammation. Once the anastomosis device is attached within the body, the anastomosis device may allow continual or temporary access into the gallbladder, according to the desired configuration.

### Anastomosis Device

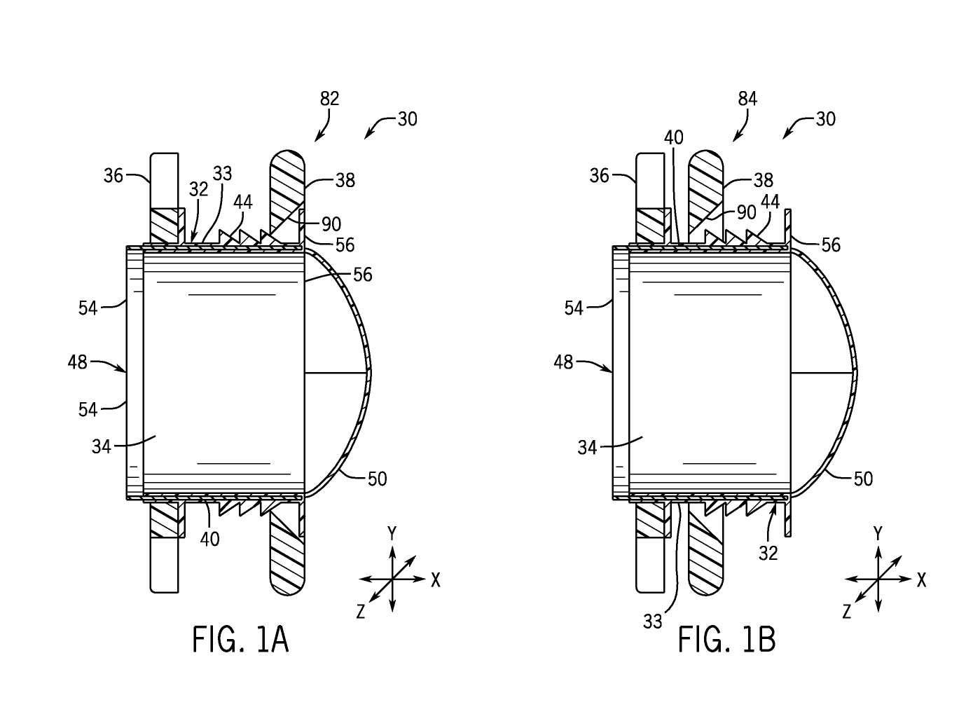

Referring now to FIGS. 1A-1B, there is shown an anastomosis device 30 according to one embodiment. As described further herein, the anastomosis device 30 may be used to cut into tissue and create an opening or connection between at least two areas (e.g., a duodenum and a gallbladder) of the body to allow material, such as gallstones, to flow between the two areas. “Cutting” may include mechanical penetration and expansion, cauterization, coring, lacerating, or incising.

The anastomosis device 30 may be sized to be delivered through at least one of a surgical port or a natural orifice of a body and through at least a portion of a gastrointestinal tract of the body of the patient to be delivered to the implantation region. As described further herein, the anastomosis device 30 may be configured to create a cauterized hole or aperture within at least one tissue wall. Accordingly, at least a portion of the anastomosis device 30 may be sized to also fit through the cauterized aperture in the tissue. For example, according to one embodiment, the length and diameter of the anastomosis device 30 may be between approximately 3 to 20 mm and 5 to 25 mm, respectively. According to another embodiment, the length and diameter of the anastomosis device 30 may be between approximately 6 to 10 mm and 7 to 13 mm, respectively.

As shown in FIGS. 1A-1B, the length, height, and width of the anastomosis device 30 may be defined by the x-axis (the longitudinal axis), y-axis (the vertical axis), and the z-axis (the lateral axis), respectively. The x-, y-, and z-axes are perpendicular to each other. Accordingly, the length of the base member 32 of the anastomosis device 30, for example, may also be defined along the x-axis of the anastomosis device 30.

The anastomosis device 30 may include a body or base member 32, which, when implanted, may extend at least partially between the duodenum and the gallbladder. The base member 32 may be a variety of different shapes. According to an embodiment, the base member 32 may be a cylindrical tube with openings on either end along the longitudinal length (the x-axis). At least a portion (e.g., the first or distal end) of the base member 32 may be sized to fit or be transported through the cauterized tissue aperture.

The base member 32 may include a base member shell 33, which may at least partially encompass a metallic ring 40 (as described further herein). The shell 33 may be constructed out of a variety of different non-conductive materials according to the desired configuration. It may be particularly beneficial for the base member shell 33 to be a non-conductive material in order to prevent the metallic ring 40 from accidentally cauterizing other areas of the body.

The base member 32 may define an inner passage or passageway 34 through the center of the anastomosis device 30. The passageway 34 may be a hollow portion of the base member 32 and may extend longitudinally along the base member 32 and between a distal or first end 54 and a proximal or second end 56 of the base member 32. The first end 54 may be used to cut the tissue and the second end 56 may be used to attach to or interface with the distal end of a delivery device, as described further herein. The passageway 34 may allow material to move between either side of the anastomosis device 30 (and thereby between two cavities within the patient, such as the duodenum and the gallbladder).

The base member 32 may include a cutting element 48 that is configured to cut tissue from the first end 54 of the base member 32 through a variety of different mechanisms. According to one embodiment, the cutting element 48 may be a cautery mechanism configured to cut the tissue through cauterization. For example, when the anastomosis device 30 is connected to the delivery device (and the delivery device is providing power to the cutting element 48, as described further herein), the cutting element 48 may act as a monopolar cautery device through the first end 54 of the base member 32. However, when the power is off or the anastomosis device 30 is disconnected from the delivery device, the cutting element 48 of the anastomosis device 30 may not cauterize or harm any tissue.

In order to transfer power or electricity from the delivery device to the first end 54 of the base member 32, the cutting element 48 may include a metallic cylinder, extension, or ring 40, located within the shell 33 of the base member 32. Power from the delivery device may be transferred from the second end 56 to the first end 54 of the anastomosis device 30 through the metallic ring 40 in order to provide power for cauterization. The power requirement to cauterize the tissue may be within a standard range supplied by a monopolar electrocautery mechanism.

The metallic ring 40 may be at least partially embedded and shielded within the base member shell 33 to prevent unintentional injury (e.g., cauterization) within the body. Accordingly, the base member shell 33 may provide insulation around the metallic ring 40. For example, the base member shell 33 may cover the inner and outer circumferential surfaces of the metallic ring 40, as well as a portion of one longitudinal end of the metallic ring 40 (corresponding to the second end 56 of the anastomosis device 30). Additional insulation may optionally be provided around the base member 32.

While the anastomosis device 30 is attached to (and powered by) the delivery device, the metallic ring 40 may only exposed through the first end 54 of the base member 32. On the first end 54 of the anastomosis device 30, the entire circumference or perimeter of the metallic ring 40 may be exposed or extend out of the base member shell 33 to allow the anastomosis device 30 controllably cauterize, and therefore cut, a complete hole or aperture through at least one tissue through the first end 54.

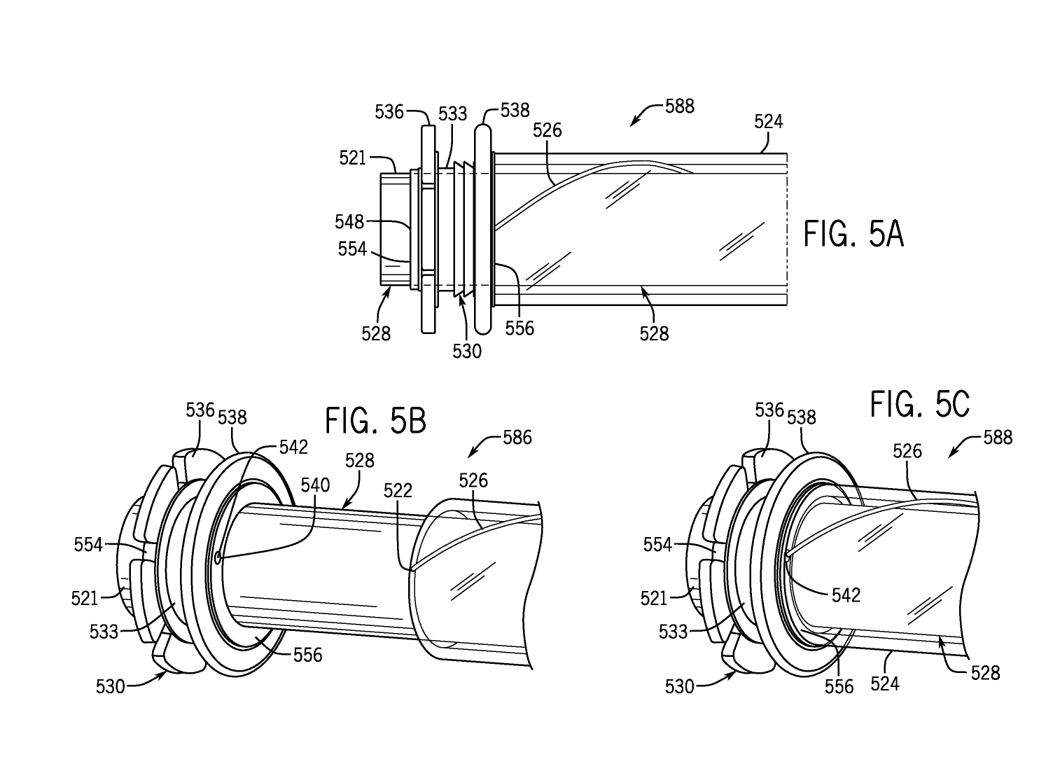

While the anastomosis device 30 is not attached to or disconnected from the delivery device or the power supply, both longitudinal ends of the metallic ring 40 may be at least partially exposed through the first end 54 of the base member shell 33 and a portion of the second end 56. For example, according to one embodiment as shown in FIG. 5B, a portion of the metallic ring 540 may be exposed through the second end 556 of the base member shell 533 when the anastomosis device 530 is disconnected from the power supply of the delivery device 528. This exposed portion of the metallic ring 540 along the second end 556 may be at least one metallic, electrical, or conductive terminal or contact 542. As shown in FIG. 5C, the contact 542 is connectable to an electrical contact from a conductive wire 526 on the delivery device 528 in order to access a power supply through the delivery device 528. The contact 542 may be a part of the metallic ring or a metallic line, area, or strip attached to the metallic ring 540.

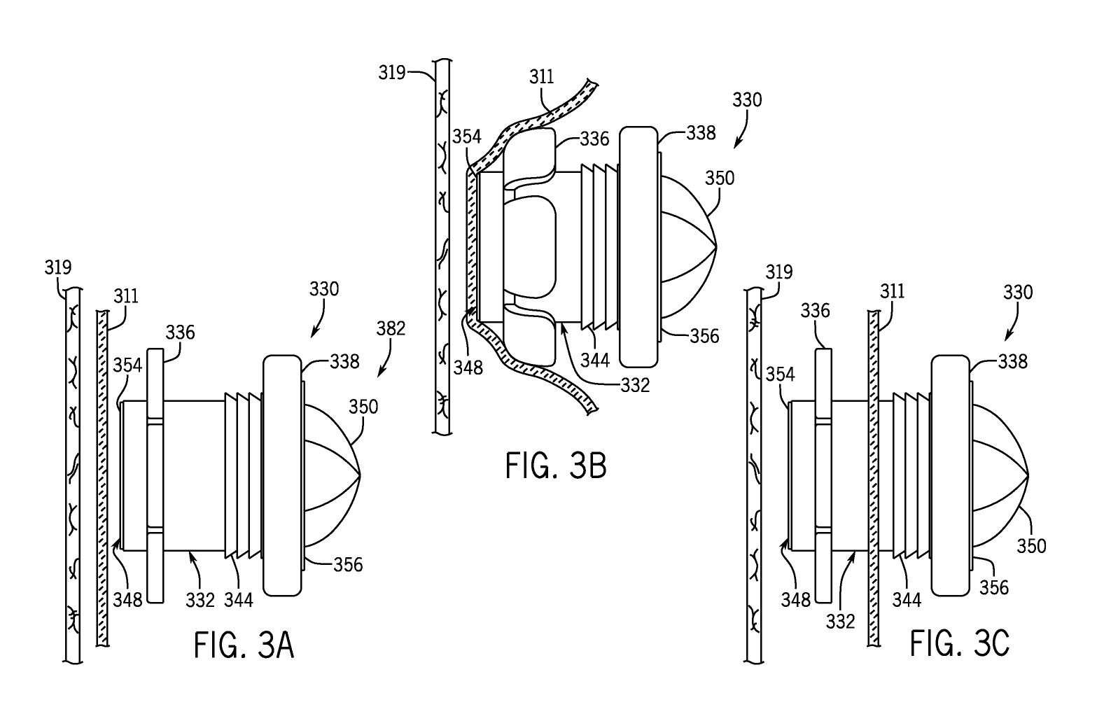

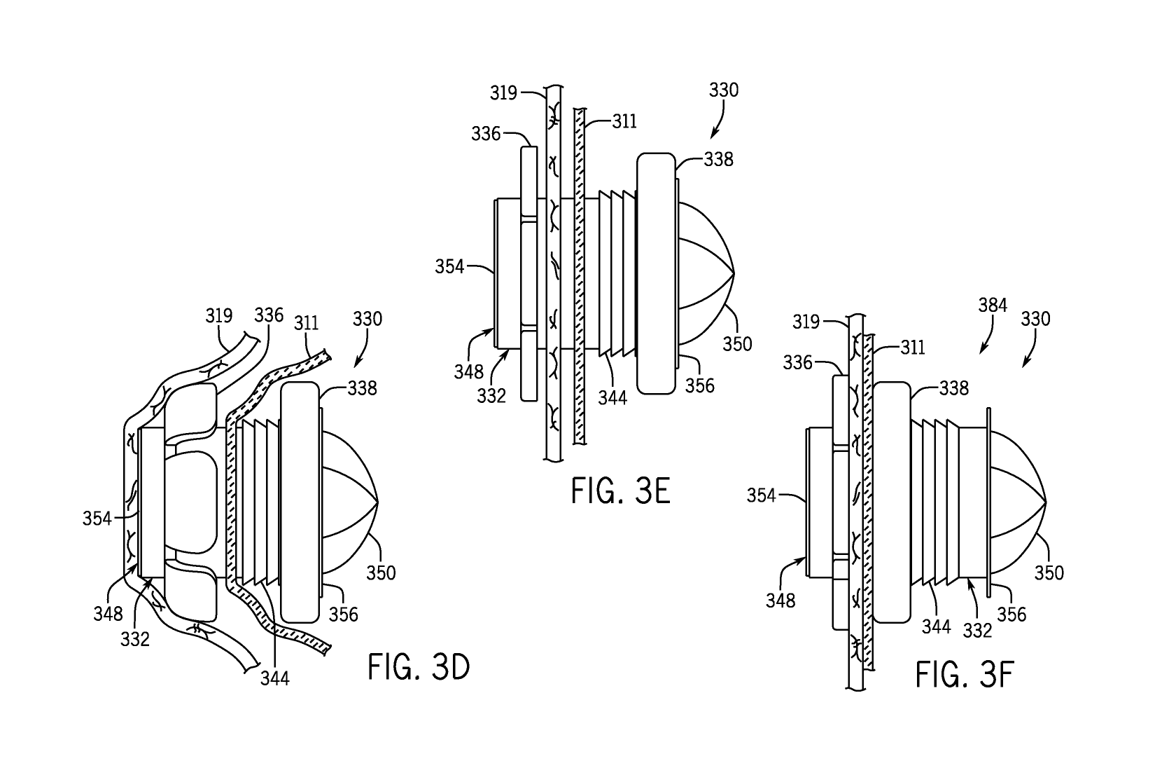

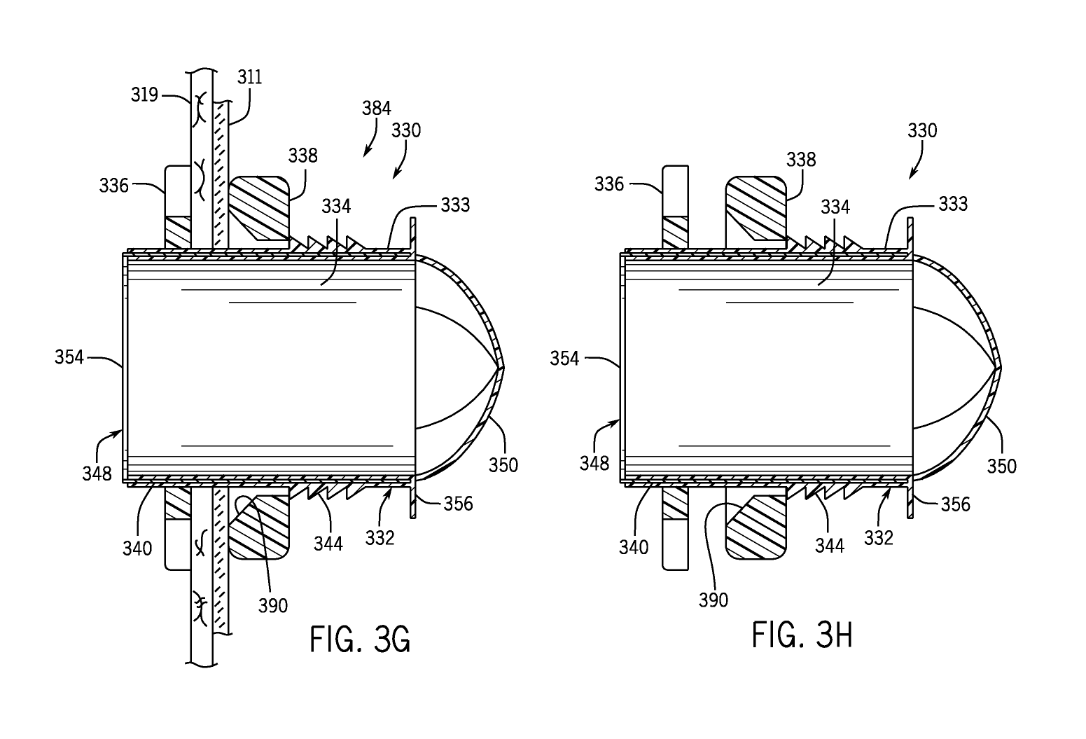

As shown in FIGS. 1A-1B, the anastomosis device 30 may further include a first flange or support member 36 and a second ring or support member 38, which may act as tissue-locking members. The support members 36 and 38 may be used to attach or secure at least one tissue wall along a portion of the base member 32 in order to anastomose the duodenum and the gallbladder. For example, according to one embodiment as shown in FIGS. 3F-3G, the support members 336 and 338 of the anastomosis device 330 may clamp or press the tissue walls 311 and 319 in between the support members 336 and 338.

As shown in FIGS. 1A-1B, the support members 36 and 38 may be coupled, connected, or attached to the base member 32 (integrally or as separate components) and may extend outwardly and radially from the base member 32. The support members 36 and 38 may be approximately perpendicular to the base member 32. In alternate embodiments, the support members may be situated at an angle to the base member 32. For example, the support members 36 and/or 38 may have an angle between approximately 30 and 60 degrees relative to the base member 32. According to another embodiment, the support members 36 and/or 38 may have an angle that is approximately 45 degrees relative to the base member 32.

The support members 36 and 38 may be proximate to each other along the length of the base member 32 (e.g., along the x-axis of the anastomosis device 30). The longitudinal distance between the support members 36 and 38 may vary according to the type and number of tissues to secure in between the support members 36 and 38. The distance between the support members 36 and 38 may be adjustable, as described further herein. The support members 36 and 38 may be shaped and sized according to the desired configuration and may have identical or mirror configurations or different configurations, as described further herein.

The first support member 36, which may be more distally located along the anastomosis device 30 than the second support member 38 and proximal to the first end 54, may be flexible or deformable in order to flex to be moved or transported through the aperture or hole the anastomosis device 30 may make in the tissue walls of both the duodenum and the gallbladder. According to one embodiment as shown in FIGS. 3B and 3D, the first support member 336 may be compressed into an undeployed state for easier delivery through an aperture within the body and may expand into a deployed state in order to maintain the position of the anastomosis device 330 within the body. The support member 336 may be preferentially compressible toward the second support member 338 and away from the cutting element 348 (as shown in FIGS. 3B and 3D) in order to prevent any tissue from slipping or falling off of the first end 354 of the anastomosis device 330.

The shape of the first support member 36 may help the first support member 36 to be moved through apertures in tissue walls, while still providing sufficient support to hold the tissue walls. For example, as shown in various embodiments (such as FIGS. 3B, 3D, 5B, and 5C), the first support member 336 or 536 may be a ring surrounded by outwardly extending flanges, petals, extensions, or separately-movable sections. As shown in FIGS. 3B and 3D, this configuration of the first support member 336 may allow the first support member 336 to be bent or compressed along the length of the base member 332 to move through the cauterized holes. The first support member 336 may be constructed out of a flexible or compressible material to allow the first support member 336 to bend or flex.

The first support member 36 may be sized to fit within the tissue aperture in the compressed state. According to one embodiment as shown in FIGS. 3A-3H, the diameter of the first support member 336 in a compressed or undeployed state (as shown, for example, in FIGS. 3B and 3D) and in an expanded or deployed state (as shown, for example, in FIGS. 3A, 3C, and 3E-3H) may be between approximately 5 to 20 mm and 10 to 30 mm, respectively. According to another embodiment, the diameter of the first support member 336 in a compressed state and an expanded state may be between approximately 10 to 15 mm and 15 to 25 mm, respectively.

As shown in FIGS. 1A-1B, the second support member 38 may be relatively more rigid or stiff, which may prevent the second support member 38 from moving through the aperture created in the tissue wall and may allow the second support member 38 to act as an anchor within the duodenum. Accordingly, the support member 38 may be a continuous ring around the base member 32 and may not be constructed out of a flexible material. The second support member 38 may be proximal to the second end 56.

At least one of the support member 36 or 38 may be movable or adjustable along the length of the base member 32 to change the distance between the support members 36 and 38 and to move the anastomosis device 30 from an undeployed position 82 (as shown in FIG. 1A) to a deployed position 84 (as shown in FIG. 1B). According to one embodiment as shown in FIGS. 1A-1B, the support member 38 may move along a portion of the length of the base member 32 (e.g., the steps 44) toward the support member 36 to move from the undeployed position 82 to the deployed position 84 and the support member 36 may remain in position along the length of the base member 32. Alternatively or additionally, the first support member 36 may be movable toward the second support member 38 and/or the second support member 38 may be stationary. This movement may allow the tissue walls to be securely clamped and/or held between the support members 36 and 38. Additionally, depending on how far the support member 36 or 38 is moved along the base member 32, the distance between the support members 36 and 38 may be easily tailored according to the desired configuration and use (e.g., to hold more tissue walls or thicker tissue walls). The support member 36 or 38 may provide traction or resistance to the other support member 36 or 38 in order to fully secure and compress the tissue between the support members 36 and 38.

As shown in FIGS. 1A-1B, the base member 32 may include an engagement structure or traction mechanism in order to secure or lock the second support member 38 (or the first support member 36) into the deployed position 84 along the base member 32. The engagement structure may be, for example, at least one discrete ridge, bump, protrusion, tooth, or snap step 44 (similar to stairs or a ladder), along a portion of the length and outside perimeter of the base member shell 33 that at least one of the support members 36 or 38 may move along. The other of the support members 36 or 38 may also move along a second set of steps (which may be angled in a different direction to direct the movement of the support member 36 or 38) or may be stationary. According to one embodiment, the base member 32 may include multiple steps 44 or a series of sequential steps 44 that the support members 36 or 38 may move along. The steps 44 may keep the support members 36 and/or 38 locked, in place, aligned, and/or in close proximity, thereby also keeping the secured tissue walls aligned and/or in close contact when the anastomosis device 30 is deployed.

The steps 44 may be angled or wedged toward one end or the center of the base member 32 to allow the support member 36 or 38 to move or slide in only one direction (e.g., unilaterally or unidirectionally) over the steps 44 along the base member 32 and to prevent the support member 36 or 38 from moving out of the deployed position 84 and away from the other support member. Once the tissue walls have been positioned between the support members 36 and 38, the support member 38 may move over the steps 44 from the second end 56 (as shown in FIG. 1A, tissue not shown) to the first end 54 and the first support member 36 (as shown in FIG. 1B, tissue not shown). Once the support member 38 has moved toward the support member 36, the support member 38 may not move backward away from the support member 36 due to the steps 44. Alternatively or additionally, the angles of the steps 44 may be positioned in different directions to direct the movement of the support members 36 or 38, according to the desired configuration.

As shown in FIGS. 1A-1B, the inner surface (or inside diameter or perimeter) of the support member 38 may have a complementary or corresponding structure or wedge 90 to the steps 44. The wedge 90 may allow the support member 38 to engage with the steps 44 by allowing movement of the support member 38 toward the support member 36 (from the undeployed position 82 to the deployed position 84) and preventing movement away from the support member 36 by locking the wedge 90 to the steps 44.

According to one embodiment as shown in FIGS. 1A-1B, the steps 44 and the wedge 90 may be angled away from each other (while in the undeployed position 82) at angles between approximately 30 to 60 degrees (and, while in the deployed position 84, the steps 44 and the wedge 90 are angled toward each other at angles between approximately 120 to 150 degrees). According to another embodiment, the steps 44 and the wedge 90 may be angled toward each other in the undeployed position 82 at approximately 45 degrees (and, in the deployed position 84, the steps and the wedge 90 are angled away from each other at approximately 135 degrees). According to another embodiment as shown in FIGS. 3G-3H, the steps 344 and the wedge 390 may be angled in the same direction.

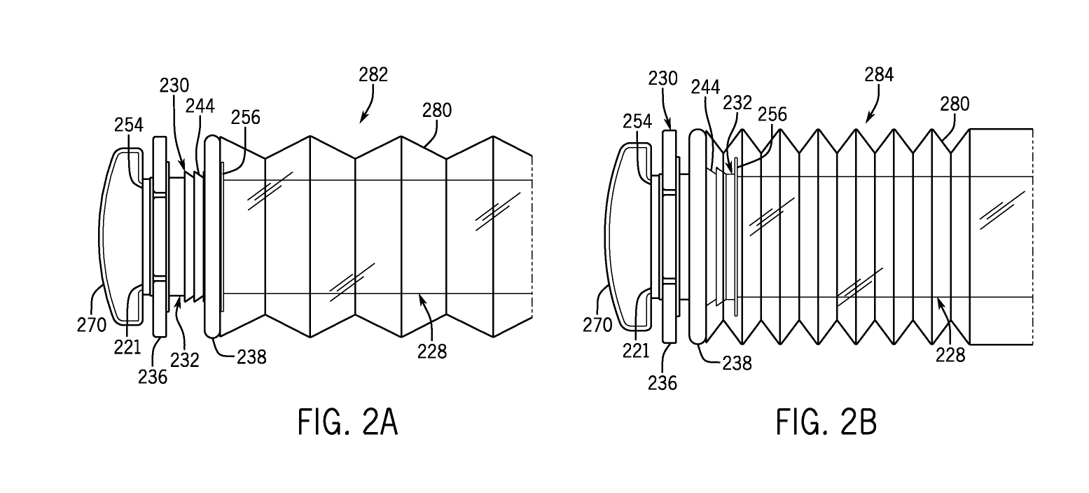

The anastomosis device 30 may optionally use pneumatic, mechanical, and/or electrical forces during and after deployment (e.g., to move the second support member 38, to clamp the tissue between the support members 36 and 38, and/or to provide a rigid attachment to the tissue walls). According to an embodiment as shown in FIGS. 2A-2B, in order to move the support members 236 and 238 toward each other and/or to secure tissue in between the support members 236 and 238 to maintain pressure on the tissue, at least one of the support members 236 and 238 may be mechanically and/or pneumatically driven or assisted. A pneumatic sleeve or bellow deployer 280 may be used to move or push the support member 238 along the base member 232. For example, the pneumatic deployer 280 may slide over the delivery device 228 and/or over a sheath with a conductive wire (as shown in FIGS. 5A-5C) and may attach to the proximal end of the support member 238. The pneumatic deployer 280 may include a channel to receive fluid (e.g., a gas or liquid). As shown in FIG. 2A, when the pneumatic deployer 280 is uninflated, the pneumatic deployer 280 may be pulled back toward the proximal end of the delivery device 228 in an undeployed position 282. In the undeployed position 282, the support member 238 may remain relatively close to the second end 256 of the anastomosis device 230. As shown in FIG. 2B, when the pneumatic deployer 280 is inflated with fluid, the pneumatic deployer 280 may move toward the distal end 221 of the delivery device 228 into a deployed position 284, which may mechanically move or push the second support member 238 along a portion of the length of the base member 232 (e.g., along the steps 244) and toward the tissue, the first end 254, and the first support member 236.

As shown in various embodiments (such as FIGS. 1A-1B and 3A-3H), an optional valve 50 or 350 may be used to control the flow of material through the anastomosis device 30 or 330 and may be attached to the base member 32 or 332. For example, the valve 50 or 350 may be attached to the first end 54 or 354 or the second end 56 or 356 of the base member 32 or 332. The valve 50 or 350 may also be used to allow the gallstones to be broken down and removed from the gallbladder through a variety of different methods and techniques, as described further herein.

According to one embodiment, the valve 50 or 350 may be a one-way valve. Accordingly, the valve 50 or 350 may be positioned to allow the bile and the gallstones to move from the gallbladder and into the duodenum and to prevent bile, gallstones, or other matter from moving back into the gallbladder.

The anastomosis device 30 may be constructed out of a variety of materials, depending on the desired configuration. For example, besides the metallic ring 40, the components of the anastomosis device 30 may be constructed out of nonconductive materials (such as non-metal material like plastic) in order to shield the patient and device from any bleed off heat and electricity and to prevent unintentional cauterization of tissue. According to one embodiment, the first support member 36 may be constructed out of a flexible material, such as an elastomer or silicon, in order to allow it to compress and flex and fit through the cauterized holes.

According to one embodiment as shown in FIGS. 3A-3H, a portion of the anastomosis device 330 may be delivered through first tissue wall 311 (e.g., the duodenum tissue wall) and the second tissue wall 319 (e.g., the gallbladder tissue wall). As shown in FIG. 3A, the anastomosis device 330 may be delivered in the undeployed position 382 to the first tissue wall 311 by a delivery device (described and shown further herein) such that the first end 354 of the anastomosis device 330 (e.g., the end with the cutting element 348) faces the first tissue wall 311. In the undeployed position 382, the first and second support members 336 and 338 may be separated along a length of the base member 332. For example, the second support member 338 may be positioned on an opposite side of the steps 344 along the base member 332 than the first support member 336.

As shown in FIG. 3B, the anastomosis device 330 may be moved toward the first tissue wall 311, such that the cutting element 348 abuts the first tissue wall 311 (e.g., the inside surface of the duodenum tissue wall). The cutting element 348 may cut the first tissue wall 311 through cauterization, which may create a hole or aperture in the first tissue wall 311. As shown in FIGS. 3G-3H, power may be transmitted to the cutting element 348 through the metallic ring 340 (from the delivery device) that extends through the base member shell 333. In order to move a portion of the anastomosis device 330 through the cauterized aperture in the first tissue wall 311, the first support member 336 bends, flexes, or compresses (due to the force of the first tissue wall 311) away from the cutting element 348 and toward the base member 332, thus decreasing the outer diameter of the anastomosis device 330.

The first support member 336, with a portion of the base member 332, is advanced through the cauterized aperture in the first tissue wall 311 and toward the second tissue wall 319 when the first support member 336 is compressed. As shown in FIG. 3C, the first support member 336 may radially expand outward from the base member 332 after the first support member 336 has been advanced through the cauterized aperture in the first tissue wall 311. The base member 332 may extend through the first tissue wall 311.

As shown in FIG. 3D, the anastomosis device 330 may be advanced toward the second tissue wall 319, such that the cutting element 348 now abuts the second tissue wall 319 (e.g., the outside surface of the gallbladder tissue wall). The cutting element 348 may cut the second tissue wall 319 through cauterization, which may create a hole or aperture in the second tissue wall 319. In order to move a portion of the anastomosis device 330 through the cauterized aperture in the second tissue wall 319, the first support member 336 bends, flexes, or compresses (due to the force of the second tissue wall 319) away from the cutting element 348 and toward the base member 332 again, thus decreasing the outer diameter of the anastomosis device 330.

The first support member 336, with a portion of the base member 32, is advanced through the cauterized aperture in the second tissue wall 319 when the first support member 336 is compressed. As shown in 3E, the first support member 336 may radially expand outward from the base member 332 after the first support member 336 has been advanced through the cauterized aperture in the second tissue wall 319. The base member 332 may extend through the first and second tissue walls 311 and 319 and the first and second support members 336 and 338 may surround the first and second tissue walls 311 and 319 along the base member 332.

As shown in FIG. 3F, the second support member 338 may be moved along the base member 332 down the steps 344 and toward the first support member 336, thus compressing the first and second tissue walls 311 and 319 toward each other between the support members 336 and 338 into the deployed position 384. The steps 344 may prevent the second support member 338 from moving away from the first support member 336. The wedge 390 (as shown in FIGS. 3G-3H) may further prevent the second support member 338 from moving away from the first support member 336. As shown in FIG. 3G, the tissue walls 311 and 319 may surround a portion of the base member 332 and be clamped or compressed together by the first and second support members 336 and 338.

As shown in FIGS. 3F-3G, the first support member 336 may directly contact the inside or mucosa of the second tissue wall 319 (e.g., the gallbladder tissue wall) and the second support member 338 may directly contact the inside or mucosa of the first tissue wall 311 (e.g., the duodenum tissue wall) in the deployed position 384. The anastomosis device 330 may directly oppose the outer or serosa surfaces of the first and second tissue walls 311 and 319, such that the respective serosa surfaces of the tissue walls 311 and 319 directly contact each other. Due to the passageway 334 of the base member 332 and the cauterized holes in the tissue walls 311 and 319, a passageway is created through the anastomosis device 330 between two organs or areas of the patient.

### Delivery Device

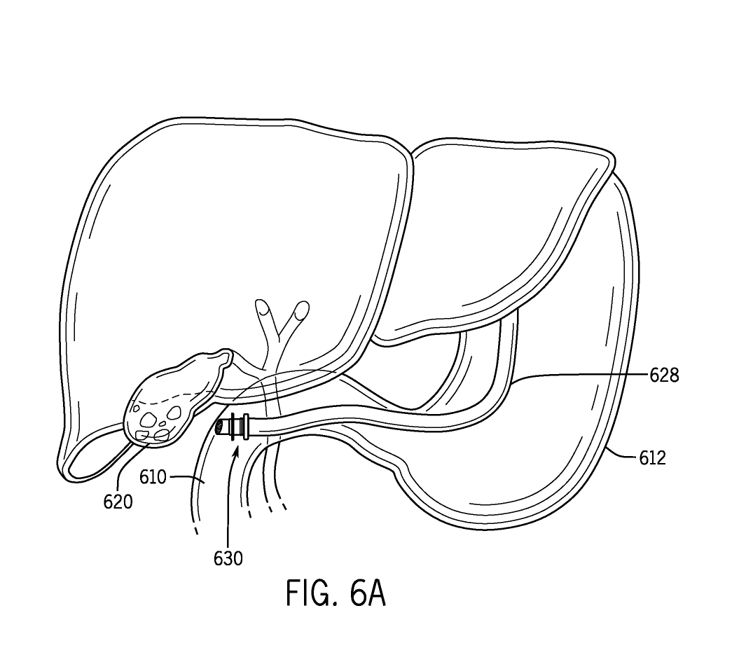

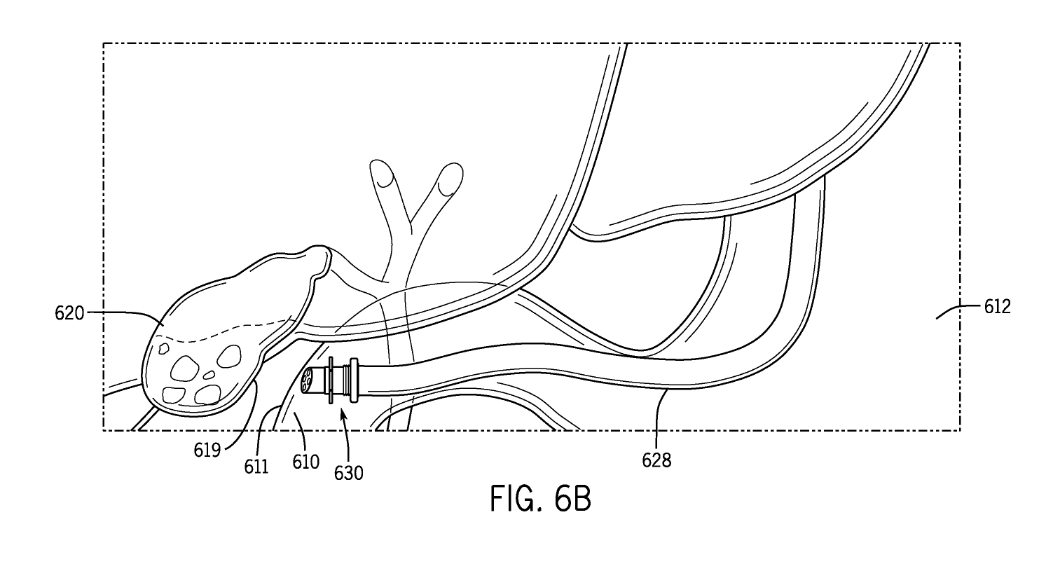

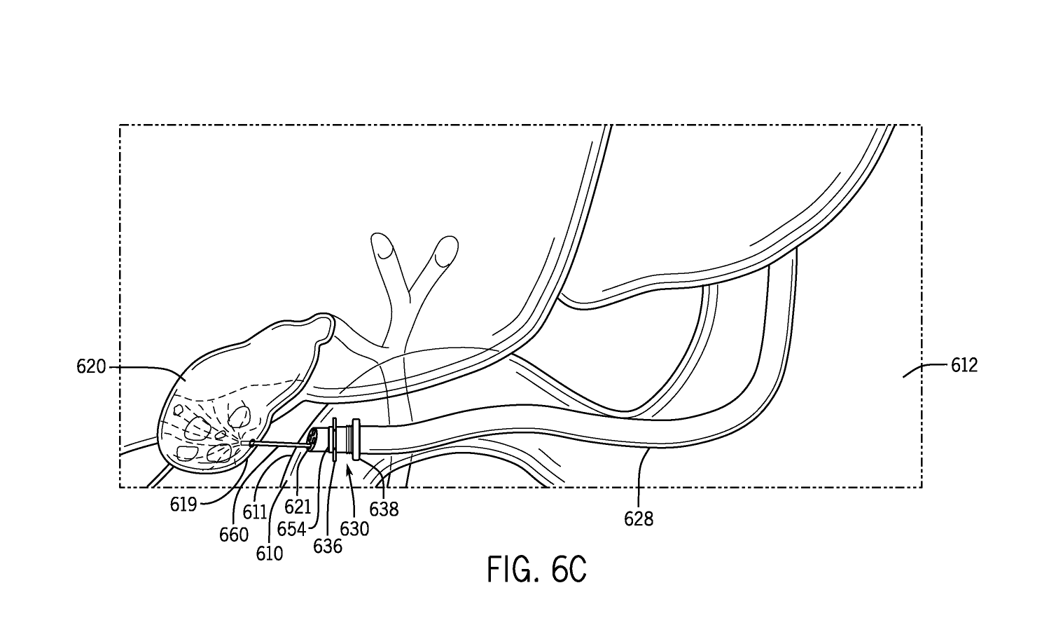

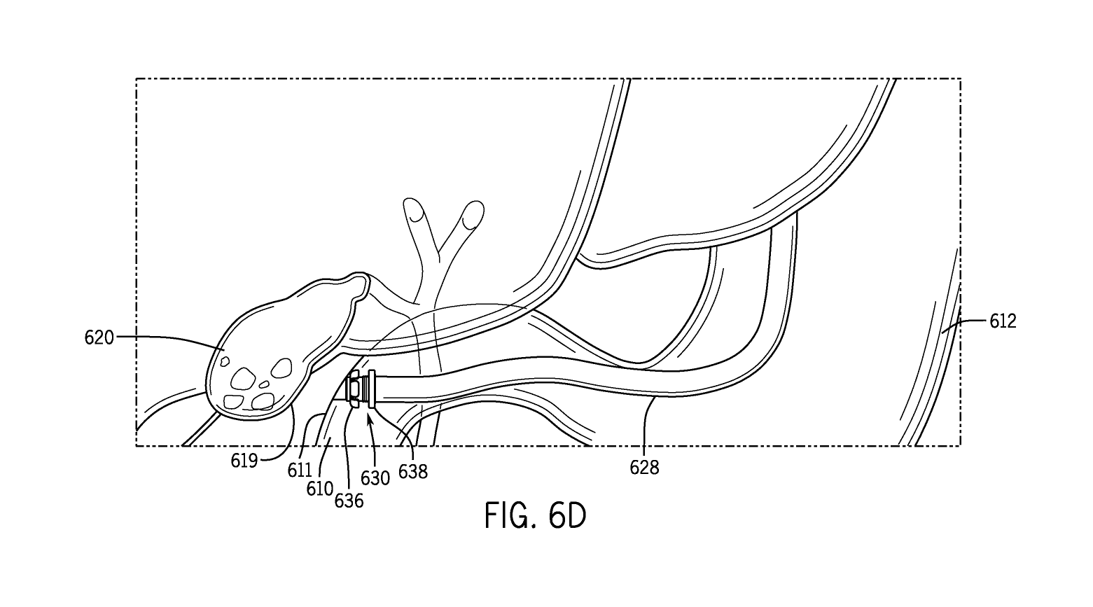

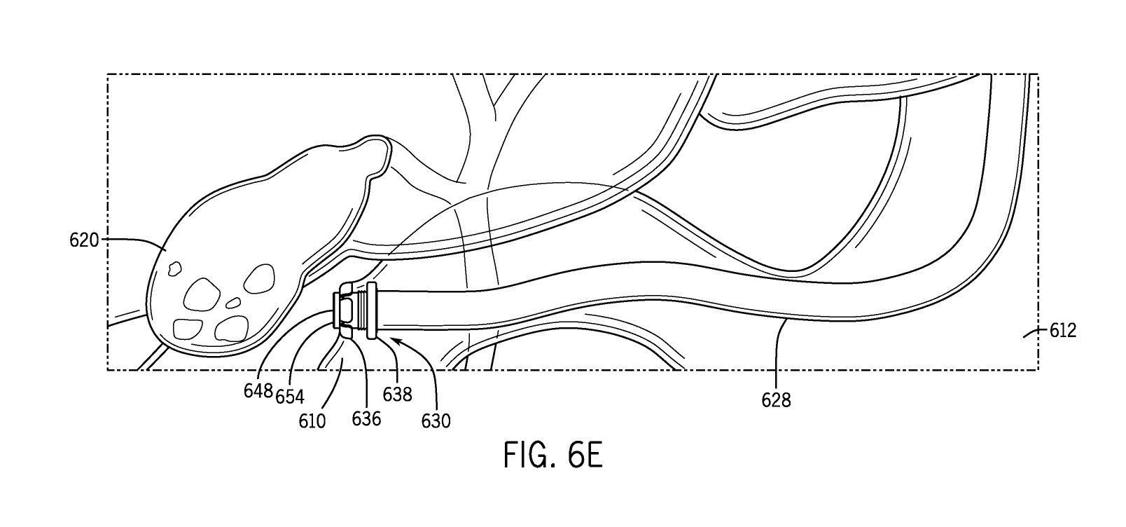

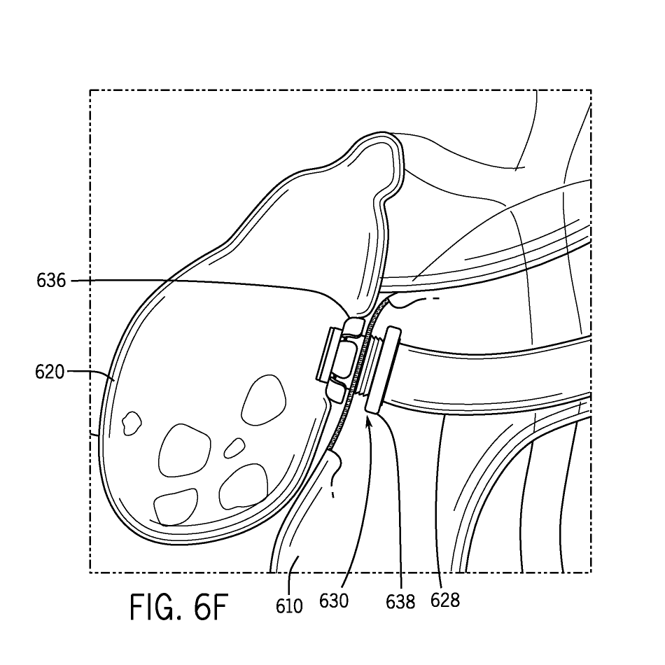

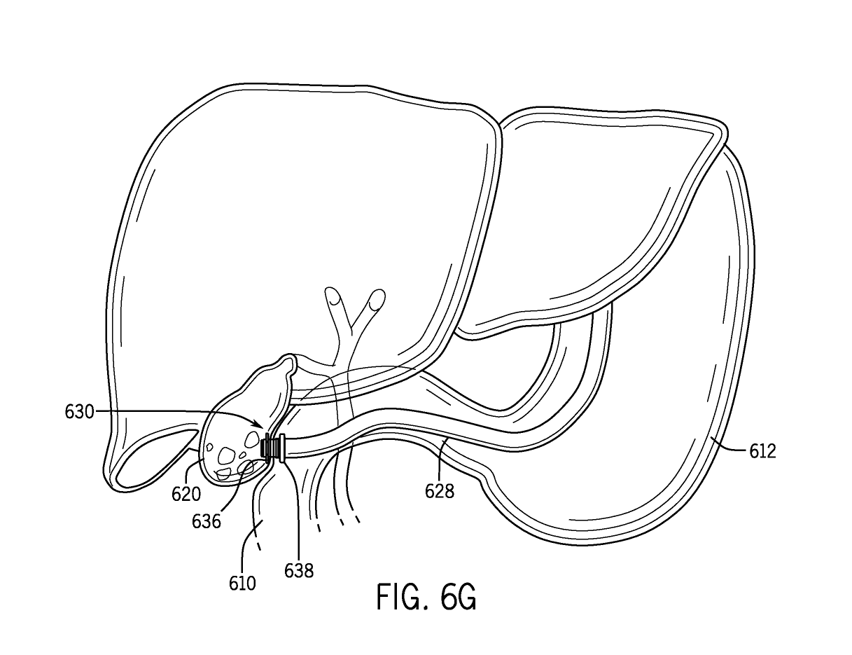

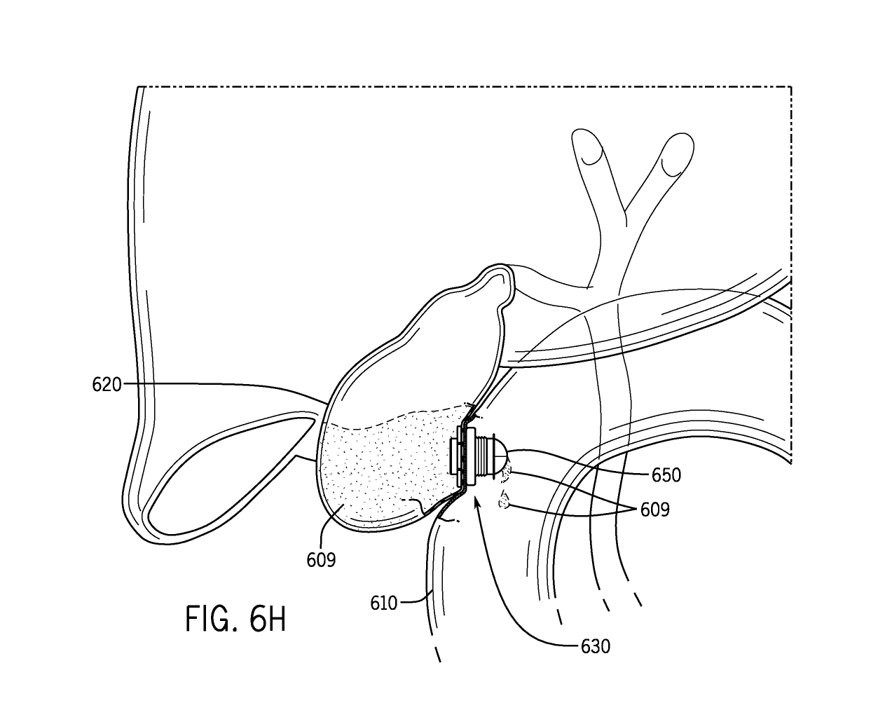

As shown in FIGS. 5A-5C, the anastomosis device 530 may attach to, be fitted to, or connect with a variety of different conventional delivery mechanisms or devices (such as a catheter or endoscope). The delivery device 528 may be designed to access the inside of the patient (as described further herein and shown in FIG. 6A) and thus may be used to advance and deliver the anastomosis device 530 to the correct location within the body of the patient, as well as provide power to the anastomosis device 530. The proximal end of the delivery device 528 refers to the end of the delivery device 528 that is configured to remain outside of the patient. The distal end 521 of the delivery device 528 refers to the end of the delivery device 528 that is configured to be inserted into the patient and to attach to the anastomosis device 530.

The delivery device 528 may be sized to fit with the anastomosis device 530 and to fit through at least one of a surgical port or natural orifice of the patient for delivery. According to one embodiment, the diameter of the delivery device 528 may be between approximately 2 to 25 mm. According to another embodiment, the diameter of the delivery device 528 may be between approximately 5 to 15 mm.

According to one embodiment, the anastomosis device 530 may be removably coupled, connected, or attached to (or near) a distal end 521 of the delivery device 528. The distal end of the delivery device 528 and the anastomosis device 530 may be sized and profiled to fit and attach with each other. As shown in FIG. 5A, the anastomosis device 530 may slide over a portion of the distal end 521 of the delivery device 528 and may be frictionally fit to the delivery device 528. Alternatively or additionally, an attachment mechanism, such as clips, snaps, or magnets (not shown), may be used to secure the anastomosis device 530 and the delivery device 528 together. The anastomosis device 530 and the delivery device 528 may remain attached during delivery and deployment (e.g., while the anastomosis device 530 is moved through the tissue walls and the second support member 538 is moved toward the first support member 536).

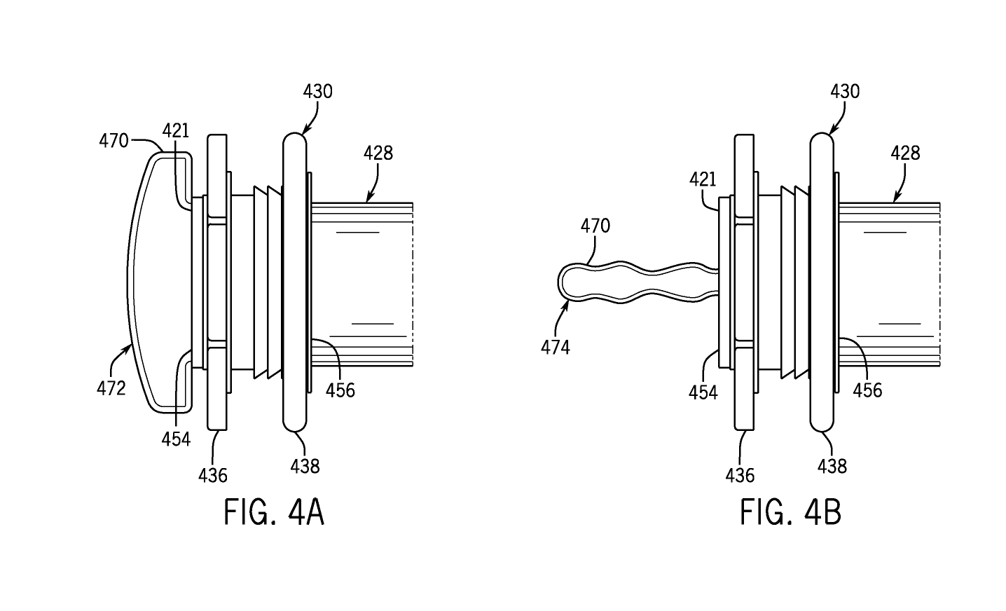

According to one embodiment as shown in FIGS. 4A-4B, in order to further secure and hold the anastomosis device 430 to the delivery device 428, a compliant supporting balloon 470 may optionally be included with or delivered through the delivery device 428. The balloon 470 may be attached to a tube (not shown) for inflation and may fit through the delivery device 428 while deflated. Once the balloon 470 is in position within the delivery device 428, the balloon 470 may be inflated to the deployed or inflated state 472 (as shown in FIG. 4A). While the balloon 470 is in the inflated state 472, the balloon 470 may prevent the anastomosis device 430 from sliding or falling off of the delivery device 428, in particular during delivery. For example, the inflated balloon 470 may extend (both laterally and radially) beyond the distal end 421 of the delivery device 428 to create a barrier along the first end 454 of the anastomosis device 430 and the distal end 421 of the delivery device 428. Alternatively or additionally, the balloon 470 may expand within a working channel of the delivery device 428 to increase the diameter of the delivery device 428 and frictionally hold the anastomosis device 430 in place along the length of the delivery device 428. FIGS. 2A-2B depict another balloon 270 included with the delivery device 228 to help secure the anastomosis device 230, according to another embodiment.

During delivery and installation, the balloon 470 may be inflated and deflated as needed to expose the first end 454 of the anastomosis device 430 in order to cauterize the tissue (in the deflated state 474) or to hold the anastomosis device 430 and the delivery device 428 together (in the inflated state 472). The balloon 470 may also provide traction to allow the second support member 438 to be pushed toward the first support member 436 without moving the anastomosis device 430 off of the delivery device 428. Accordingly, the second end 456 of the anastomosis device 430 may remain attached to the delivery device 428 during delivery and deployment.

In order to remove the delivery device 428 from the anastomosis device 430 after the anastomosis device 430 has been delivered, positioned, and clamped to the tissue, the balloon 470 may be deflated into the undeployed or deflated state 474 (as shown in FIG. 4B) and the delivery device 428 may be removed from within the anastomosis device 430, leaving the anastomosis device 430 in position within the body of the patient.

As shown in FIGS. 5A-5C, the delivery device 528 may provide power to certain portions of the anastomosis device 530, such as the cutting element 548, in order to cauterize the tissue. The delivery device 528 may include an electrical, metallic or conductive line, strip, trace, lead, or wire 526 that extends along the length of the delivery device 528 to transmit power or carry a current from an outside power source (outside of the patient) at the proximal end of the delivery device 528 to an electrical contact 522 toward the distal end 521 of the delivery device 528. As shown in FIGS. 5B-5C, the electrical contact 522 on the delivery device 528 is connectable with the electrical contact 542 on the anastomosis device 530 to provide power to the cutting element 548 located on the first end 554 of the anastomosis device 530.

According to one embodiment as shown in FIGS. 5A-5C, the conductive wire 526 may be housed or embedded in a separate, flexible sleeve or sheath 524 that is positionable outside of and adjacent to the body of the delivery device 528. The sheath 524 may slide over the delivery device body to contact and thus provide power to the anastomosis device 530. According to other embodiments, the conductive wire 526 may be a part of the delivery device 528 or may be inserted through and housed in a separate, designated, and/or working channel within the delivery device 528. For example, the delivery device 528 may utilize an endoscopic channel to carry or transmit current or power to the distal end 521 of the delivery device 528. According to another embodiment, the conductive wire 526 (with insulation) may run adjacent to the outer surface of the delivery device 528 without a sheath 524. According to another embodiment, the anastomosis device 530 may be powered through induction or wireless charging and the anastomosis device 530 or the delivery device 528 may include a receptor. The sheath 524 and/or the various channels or layers of the delivery device 528 may be non-conductive to provide shielding to the conductive wire 526 and prevent any harm to surrounding tissue within the body.

FIGS. 5B-5C depict the sheath 524 moving from an unattached position 586 (where the sheath and the anastomosis device 530 are not directly connected) to an attached position 588 such that the sheath 524 and the anastomosis device 530 are attached to transmit power to the cutting element 548. As shown in FIG. 5B, the distal end of the sheath 524 may have at least one exposed metallic, conductive, or electrical terminal or contact 522 configured to align with, contact, directly interface, and transmit power to the electrical contact 542 on the second end 556 of the anastomosis device 530. Alternatively, the distal end 521 of the delivery device 528 have include the exposed contact 522 to connect with the electrical contact 542 on the anastomosis device 530. The contact 522 may correspond to or be complementary to the electrical contact 542 on the second end of the anastomosis device 530 such that the contacts 522 and 542 may be in direct contact to transmit electricity. While the anastomosis device 530 and the delivery device 528 are electrically attached, the respective contact areas are attached and aligned to each other (as shown in FIGS. 5A and 5C) and may be shielded by each other. When the power is turned on and the anastomosis device 530 is attached to the delivery device 528, electric current (e.g., electrocautery power) may be transmitted from the contact 522 on the delivery device 528 to the contact 542 on the anastomosis device 530 to power the cutting element 548.

The sheath 524 and the second end 556 of the anastomosis device 530 may attach to each other through a variety of different mechanisms, including but not limited to snaps, clips, and magnets. As shown in FIG. 5B, the sheath 524 may be retracted or disconnected from the anastomosis device 530, which may prevent any power from being transmitted to the anastomosis device 530. The sheath 524 may slide over the body of the delivery device 528 to attach to and electrically connect with the anastomosis device 530 (as shown in FIGS. 5A and 5C), such that the anastomosis device 530 may be powered.

As described further herein and shown in FIGS. 2A-2B, the delivery device 228 may include an additional outer sleeve, such as the pneumatic deployer 280, which may be used to move at least one support member 236 or 238 along the length of the base member 232 of the anastomosis device 230.

### Gallstone Removal

FIGS. 6A-H depict the anastomosis device 630 being deployed and implanted within a patient to allow the gallbladder 620 to drain into the duodenum 610 according to one embodiment. In order to deploy the anastomosis device 630 within the body to access the gallbladder 620 and remove gallstones from the body, the anastomosis device 630 may first be attached to or near the distal end of the delivery device 628. The delivery device 628 (along with the anastomosis device 630) may be inserted through a surgical port or a natural orifice of the body of the patient, such as the mouth. The delivery device 628 may be progressed or advanced through the gastrointestinal tract (e.g., through the esophagus and into the stomach 612) to the duodenum 610, as shown in FIGS. 6A-6B.

Once the delivery device 628 has positioned the anastomosis device 630 proximal to the duodenum tissue wall 611 within the duodenum 610, the gallbladder 620 may be cleaned and rinsed using endoscopic suction and irrigation techniques. As shown in FIG. 6C, a needle 660 may be used to prevent any obstructions from impeding the removal and clearing of the gallstones. The needle 660 may be coupled or connected to the delivery device 628 and may extend or advance from a port on the distal end 621 of the delivery device 628 (such as an endoscopic port) and forwardly through the first end 654 of the anastomosis device 630. As the needle 660 is extended through the distal end 621 of the delivery device 628, the needle 660 may pierce through the duodenum tissue wall 611 and the gallbladder tissue wall 619 and insert at least partially into the gallbladder 620. The needle 660 may optionally be positioned and inserted between approximately 70-110° to the tissue walls. (According to one embodiment, the needle 660 may be inserted at approximately 90° to the tissue wall.) Once the needle 660 is positioned at least partially within the gallbladder, the needle 660 may clean, rinse, and/or irrigate the gallbladder 620 with, for example, saline solution. The needle 660 may also provide suction to remove material, matter, and/or the saline from the gallbladder 620 and to ensure that the sides of the gallbladder 620 (along the inside of the gallbladder tissue wall 619) are cleared.

After the gallbladder 620 has been cleaned and/or rinsed, the needle 660 may be retracted back into the port of the delivery device 628. After the needle 660 is retracted within or covered by the delivery device 628, the delivery device 628 may be extended toward the duodenum tissue wall 611, as shown in FIG. 6D. According to another embodiment, the needle 660 may remain the gallbladder 620 as the anastomosis device 630 is deployed in order to continuously clear the area (e.g., the gallbladder 620) during the procedure (not shown).

The anastomosis device 630 may be advanced toward the duodenum 610, such that the first end 654 of the anastomosis device 630 is flush with the duodenum tissue wall 611. (According to another embodiment, if the anastomosis device 630 is not already at the distal end 621 of the delivery device 628, the anastomosis device 630 may be progressed along the delivery device 628 to the distal end 621 of the delivery device 628.) Additional pressure may be exerted to ensure that the anastomosis device 630 has complete contact with the duodenum tissue wall 611. The first support member 636 may begin to abut the duodenum tissue wall 611, causing the first support member 636 to bend.

Once the anastomosis device 630 is properly positioned and abuts the inside surface of the tissue wall 611 of the duodenum 610, the duodenum tissue wall 611 (and, subsequently, the gallbladder tissue wall 619) may be cut. For example, the cutting element 648 on the first end 654 of the anastomosis device 630 may cut or form an aperture in the tissue walls with at least one of cauterization (e.g., electrocautery or ultrasound cauterization), perforation, radio frequency cutting, mechanical cutting, laser cutting, or harmonic cutting. The electrocautery may optionally be monopolar or bipolar.

To power the anastomosis device 630 (as shown and described in FIGS. 5A-5C), the delivery device 628 may transmit a current along the length of the delivery device 628 to an electrical contact on the anastomosis device 630. The electrical contact may subsequently transmit the current through the metallic ring (as described further herein) and toward the cutting element 648 to allow the cutting element 648 to cut the tissue wall.

Since the distal, exposed, the cutting element 648 is in direct contact with the tissue wall, the anastomosis device 630 may cauterize the tissue wall to create a hole or aperture. Once a hole has been made in the duodenum tissue wall 611, the anastomosis device 630 may be advanced at least partially through the cauterized hole in the duodenum 610 and beyond the duodenum 610 (toward the gallbladder 620), as shown in FIG. 6E. The first support member 636 may also be advanced through the duodenum tissue wall 611 with a portion of the base member of the anastomosis device 630, while the second support member 638 may remain within the duodenum 610 to act as an anchor.

The anastomosis device 630 may be further advanced toward the tissue wall of the gallbladder 620 and may be positioned such that the cutting element 648 the anastomosis device 630 is in direct contact with or abuts the outside surface of the gallbladder tissue wall 619 in order for the anastomosis device 630 to cut an aperture in the tissue wall through cauterization, as described further herein. Once the aperture has been cut in the gallbladder tissue wall 619, the anastomosis device 630 may have access to the inside of the gallbladder 620 and may be progressed at least partially into the gallbladder 620, as shown in FIG. 6F. The first support member 636 may also be moved through the aperture in the gallbladder 620, while the second support member 638 may remain in the duodenum 610, as shown in 6G.

Once the anastomosis device 630 is in position within both the duodenum 610 and the gallbladder 620, the second support member 638 may be moved along the base member of the anastomosis device 630 toward the first support member 636 (as shown in various embodiments (such as FIGS. 1A-1B and 3A-3H) and described further herein), which may clamp the tissue walls 611 and 619 between the support members 636 and 638 around an outer circumference or perimeter of the base member, thereby deploying the anastomosis device 630, securing the anastomosis device 630 to the tissues, providing a passageway between the duodenum 610 and the gallbladder 620, and anastomosing the duodenum 610 and the gallbladder 620.

Once the anastomosis device 630 is in place, the inside of the gallbladder 620 may be easily accessed. Due to the strong grip of the anastomosis device 630 on the tissue walls 611 and 619, the anastomosis device 630 prevents any gaps between the tissue walls, which prevents any leakage of bile, thus preventing any chance of inflammation or contamination.

The gallstones may be broken down through a variety of different methods. According to one embodiment, the gallstones may be mechanically broken by introducing various medical devices through a port or working channel in the delivery device 628 and through the anastomosis device 630. Alternatively or additionally, the gallstones may be broken up with lasers, shockwaves, and/or chemicals (e.g., medicine). The delivery device 628 may be also be used to further clean, irrigate, and suction the gallbladder 620 through a port of the delivery device 628. As shown in FIG. 6G, the delivery device 628 may remain attached to the deployed anastomosis device 630 in order to allow additional or further procedures to be conducted (to, for example, treat or clear the gallstones).

The gallbladder 620 may be drained and/or the gallstones may be removed from the gallbladder 620 through the base member of the anastomosis device 630 and into either the duodenum 610 (and subsequently be excreted from the body through the digestive system) or through the delivery device 628 (and thus removed from the body).

As shown in FIG. 6H, the second support member 638 may be moved toward the first support member 636 to secure the tissue walls of the duodenum 610 and the gallbladder 620 together. The delivery device 628 may be detached from the anastomosis device 630 and removed from the body, leaving the anastomosis device 630 in place within the patient. The anastomosis device 630 may continue to anastomose the duodenum 610 and the gallbladder 620 and may allow material or matter 609 (e.g., gallstones and/or bile) to move from the duodenum 610 to the gallbladder 620 through a passageway within the anastomosis device 630. For example, FIG. 6H depicts matter 609 (e.g., bile) dripping from the gallbladder 620, through the valve 650, and into the duodenum 610, which may prevent recurrent gallstones from forming. The anastomosis device 630 may be left in place within the body either temporarily or permanently.

The entire procedure may optionally be internally visualized continuously through, for example, an endoscopic visual camera, ultrasound computed tomography, magnetic resonance imaging guidance, or fluoroscopic guidance. Alternatively or additionally, the procedure may be externally visualized through, for example, radiopaque imaging under ultrasound or computed tomography. A dye may or may not be used with the fluoroscopic guidance.

Although the duodenum and gallbladder are referred to, it is anticipated that the anastomosis device may attach to and provide a passageway through any tissue walls and may connect or anastomose any combination of hollow or filled cavities, members, organs, structures, lumens, tubes, cysts, tissues, or areas of the body, according to the desired use. For example, the anastomosis device may be configured to anastomose any combination of the stomach, jejunum, duodenum, ilium, gallbladder, cystic ducts, bile ducts, common bile duct, biliary system, a pseudocyst of a pancreas, or a liver. Further, multiple cavities may be connected to each other and multiple tissue walls may be secured between the first and second support members. Although the gastrointestinal tract is referred to, the anastomosis device may be used within any area or tract of the body.

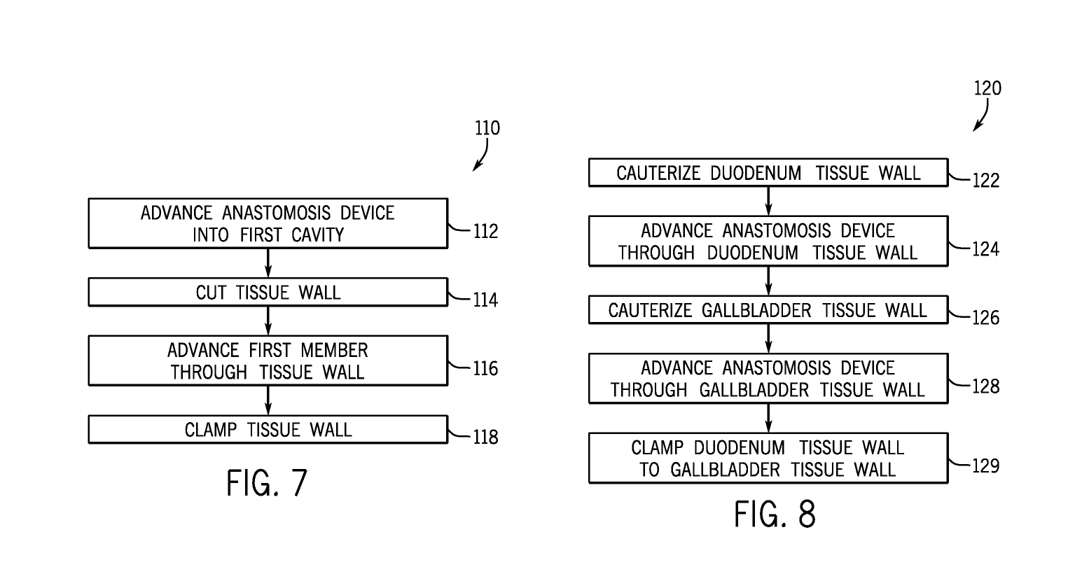

Referring now to FIG. 7, a anastomosis method 110 is shown. An anastomosis device may be advanced into a first cavity (112). The anastomosis device may include a body defining an inner passage between a first end and a second end. A first member and a second member may extend outwardly from the body of the anastomosis device. A tissue wall may be cut with a cutting element on a first end of the anastomosis device (114), which may create an aperture in the tissue wall. Multiple tissue walls may be cut according to the desired use. The first member of the anastomosis device may subsequently be advanced through the at least one tissue wall to a second cavity (116) and the second member may remain in the first cavity. The at least one tissue wall may be clamped between the first member and the second member (118) by, for example, moving at least one of the first or second members toward the other.

Referring now to FIG. 8, a method 120 for accessing a gallbladder is shown. A duodenum tissue wall may be cauterized (122) with, for example, a cutting element on a first end of an anastomosis device to create an aperture in the duodenum tissue wall. The body of the anastomosis device may define a passageway through the anastomosis device. The anastomosis device may be advanced at least partially through the duodenum tissue wall (124). The anastomosis device may be advanced toward the gallbladder and a gallbladder tissue wall may be cauterized with the cutting element of the anastomosis device (126) to create an aperture in the gallbladder tissue wall. Subsequently, the anastomosis device may be advanced at least partially through the gallbladder tissue wall (128). The duodenum tissue wall and the gallbladder tissue wall may be clamped together (129) around an outer circumference between the first end and a second end of the anastomosis device.

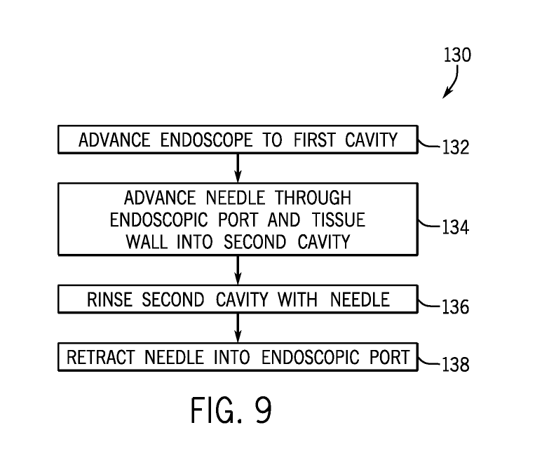

Referring now to FIG. 9, a method 130 for cleaning a cavity within a body is shown. An endoscope may be advanced to a first cavity within the body (132). A distal end of the endoscope may include an endoscopic port and the first cavity may include a tissue wall. A needle may be advanced through the endoscopic port and the tissue wall into a second cavity (134). Subsequently, the second cavity may be rinsed, cleaned, and/or drained with the needle (136). The needle may then be refracted back into the endoscopic port (138).

The embodiments disclosed herein anastomose two areas of the body and allow material to move between the cavities. Besides those embodiments depicted in the figures and described in the above description, other embodiments are also contemplated.

It is anticipated that the various components, configurations, systems, methods, and features of the different embodiments of the anastomosis device may be combined or used alone according to the desired use and configuration.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### Various Embodiments

According to one embodiment, an anastomosis device may include a base member defining a passageway between a first end and a second end of the base member, a first support member connected to the base member proximal to the first end, and a second support member connected to the base member proximal to the second end. The base member may have a cutting element to cut tissue from the first end. At least one of the first support member and the second support member may be movable along a length of the base member to secure a portion of tissue between the first and second support members.

In any of the embodiments, the base member may be releasably engagable to a distal end of a delivery device.

In any of the embodiments, the base member may be configured to slide over the distal end of the delivery device.

In any of the embodiments, the anastomosis device may include a supporting balloon that is inflatable and deflatable at the distal end of the delivery device. The supporting balloon may prevent the base member from detaching from the delivery device when inflated.

In any of the embodiments, the delivery device may be an endoscope.

In any of the embodiments, the cutting element may be powered by the delivery device.

In any of the embodiments, the delivery device may include a separate channel to house an electrical line that is configured to carry a current.

In any of the embodiments, current may be transmitted through a conductive delivery device contact on the delivery device to a conductive anastomosis contact on the second end of the base member when the conductive delivery device contact and the conductive anastomosis contact are in direct contact.

In any of the embodiments, the anastomosis device may include a needle connected to the delivery device and configured to be extended forwardly from the base member.

In any of the embodiments, the anastomosis device may include a sheath with an electrical line extending along the sheath and configured to transmit power from a proximal end of the delivery device to the distal end of the delivery device. The sheath may be configured to slide over the delivery device to contact the second end of the base member.

In any of the embodiments, the first support member and the second support member may be configured to clamp together tissue from at least two organs to anastomose the at least two organs.

In any of the embodiments, the two organs may be a duodenum and a gallbladder.

In any of the embodiments, the first support member may abut a tissue wall of the gallbladder and the second support member may abut a tissue wall of the duodenum.

In any of the embodiments, the diameter of the base member may be between 7 to 13 mm.

In any of the embodiments, a shell of the base member may provide insulation around the cutting element. The cutting element may be exposed through the shell along the first end of the base member.

In any of the embodiments, the cutting element may utilize at least one of cauterization, perforation, monopolar or bipolar electrocautery, ultrasound cauterization, radio frequency cutting, mechanical cutting, laser cutting, or harmonic cutting.

In any of the embodiments, the first support member and the second support member may be configured to clamp a tissue wall of a gallbladder and a tissue wall of a duodenum therebetween.

In any of the embodiments, the first support member may be deformable.

In any of the embodiments, the first support member may include an elastomer.

In any of the embodiments, the first support member may include silicon.

In any of the embodiments, the base member may include an engagement structure along at least a portion of an outside perimeter of a shell of the base member and at least one of the first support member or the second support member may be movable along the engagement structure.

In any of the embodiments, the engagement structure may be a series of sequential steps angled toward a center of the base member to allow at least one of the first support member or the second support member to move in one direction along the outside perimeter of the shell.

In any of the embodiments, an inner circumference of one of the first support member or the second support member may include at least one wedge that is complementary to the steps.

In any of the embodiments, the anastomosis device may include a pneumatic deployer configured to push one of the first support member and the second support member along the base member when inflated.

In any of the embodiments, the anastomosis device may include a one-way valve attached to the base member.

In any of the embodiments, the base member may be configured to provide a passage between at least one of two cavities, tissues, lumens, tubes, organs, hollow members, or cysts.

According to another embodiment, an anastomosis method may include advancing an anastomosis device into a first cavity with a first tissue wall, cutting the first tissue wall with the first end of the anastomosis device, advancing the first support member through the first tissue wall to a second tissue wall of a second cavity, cutting the second tissue wall with the first end of the anastomosis device, advancing the first support member through the second tissue wall, and clamping the first and second tissue walls between the first support member and the second support member. The anastomosis device may include a base member defining an inner passage between a first end and a second end, and a first support member and a second support member extending outwardly from the base member.

In any of the embodiments, the step of cutting the first and second tissue walls may include cauterizing the first and second tissue walls.

In any of the embodiments, the anastomosis method may include delivering the anastomosis device through at least one of a surgical port or a natural orifice of a patient.

In any of the embodiments, the anastomosis method may include delivering the anastomosis device through at least a portion of the gastrointestinal tract of the patient.

In any of the embodiments, the anastomosis method may include providing a passage through the anastomosis device between the first cavity and the second cavity.

In any of the embodiments, the anastomosis method may include using pneumatic forces to move the second support member toward the first support member along the base member to clamp the first and second tissue walls between the first support member and second support member.

In any of the embodiments, the anastomosis method may include moving the second support member along a length of the base member.

In any of the embodiments, the anastomosis method may include releasably attaching the anastomosis device to an endoscope and advancing the anastomosis device with the endoscope.

In any of the embodiments, the anastomosis method may include inflating a supporting balloon to maintain the attachment between the anastomosis device and the endoscope and deflating the supporting balloon to release the anastomosis device from the endoscope.

In any of the embodiments, the anastomosis method may include powering the anastomosis device with the endoscope.

In any of the embodiments, the step of powering the anastomosis device may include transmitting a current from a conductive endoscopic contact on the endoscope to a conductive anastomosis contact on the second end of anastomosis device when the conductive endoscopic contact and the conductive anastomosis contact are in direct contact.

In any of the embodiments, the anastomosis method may include providing a one-way valve on the second end of the anastomosis device.

In any of the embodiments, the anastomosis method may include cleaning the second cavity with a needle prior to cutting the first tissue wall.

In any of the embodiments, the anastomosis method may include guiding the anastomosis device with fluoroscopic guidance.

In any of the embodiments, the anastomosis method may include using a dye with the fluoroscopic guidance.

In any of the embodiments, the anastomosis method may not using a dye with the fluoroscopic guidance.

In any of the embodiments, the anastomosis method may include using at least one of ultrasound computed tomography or magnetic resonance imaging guidance.

In any of the embodiments, the anastomosis method may include anastomosing the first cavity and the second cavity.

In any of the embodiments, the anastomosis method may include anastomosing two organs in the gastrointestinal tract of a patient.

According to yet another embodiment, a method for accessing a gallbladder may include cauterizing a duodenum tissue wall of a duodenum with a first end of an anastomosis device, advancing the anastomosis device at least partially through the duodenum tissue wall, cauterizing a gallbladder tissue wall of the gallbladder with the first end of the anastomosis device, advancing the anastomosis device at least partially through the gallbladder tissue wall, and clamping the duodenum tissue wall to the gallbladder tissue wall around an outer circumference of the base member between the first end and the second end. A base member of the anastomosis device may define a passageway through the anastomosis device from the first end to the second end of the anastomosis device. The anastomosis device may at least partially remain in the duodenum.

In any of the embodiments, the method for accessing a gallbladder may include at least one of draining, removing stones from, or performing a procedure on a gallbladder.

In any of the embodiments, the method for accessing a gallbladder may include delivering the anastomosis device through at least one of a surgical port or a natural orifice of a patient.

In any of the embodiments, the method for accessing a gallbladder may include delivering the anastomosis device through at least a portion of the gastrointestinal tract of the patient.

In any of the embodiments, the method for accessing a gallbladder may include providing a passage between the gallbladder and the duodenum.

In any of the embodiments, the method for accessing a gallbladder may include moving at least one of a first support member and a second support member along a length of the base member. The first support member and the second support member may be configured to clamp the duodenum tissue wall and the gallbladder tissue wall together.

In any of the embodiments, the method for accessing a gallbladder may include using at least one of pneumatic, mechanical, and electrical forces to move at least one of the first support member and the second support member.

In any of the embodiments, the method for accessing a gallbladder may include releasably attaching the anastomosis device to an endoscope and advancing the anastomosis device with the endoscope.

In any of the embodiments, the method for accessing a gallbladder may include inflating a supporting balloon to maintain the attachment between the anastomosis device and the endoscope and deflating the supporting balloon to release the anastomosis device from the endoscope.

In any of the embodiments, the method for accessing a gallbladder may include powering the anastomosis device with the endoscope.

In any of the embodiments, the step of powering the anastomosis device may include transmitting a current from a conductive endoscopic contact on the endoscope to a conductive anastomosis contact on the second end of anastomosis device when the conductive endoscopic contact and the conductive anastomosis contact are in direct contact.

In any of the embodiments, the method for accessing a gallbladder may include providing a one-way valve on the second end of the anastomosis device.

In any of the embodiments, the method for accessing a gallbladder may include cleaning the gallbladder with a needle prior to cauterizing the duodenum and the gallbladder.

In any of the embodiments, the method for accessing a gallbladder may include guiding the anastomosis device with fluoroscopic guidance.

In any of the embodiments, the method for accessing a gallbladder may include using at least one of ultrasound computed tomography or magnetic resonance imaging guidance.

In any of the embodiments, the method for accessing a gallbladder may include anastomosing the gallbladder and the duodenum.

According to still another embodiment, a method for cleaning a cavity within a body may include advancing an endoscope to a tissue wall of a first cavity within the body, advancing a needle through the endoscopic port and the tissue wall into a second cavity, rinsing the second cavity with the needle, and retracting the needle into the endoscopic port. A distal end of the endoscope may include an endoscopic port.

In any of the embodiments, the step of rinsing the second cavity may include irrigating the second cavity.

In any of the embodiments, the method for cleaning a cavity may include rinsing the second cavity with a saline solution.

In any of the embodiments, the method for cleaning a cavity may include clearing matter along a tissue wall of the second cavity.

In any of the embodiments, the method for cleaning a cavity may include using endoscopic suction to clean the second cavity.

In any of the embodiments, the method for cleaning a cavity may include cleaning the second cavity continuously.

In any of the embodiments, the method for cleaning a cavity may include anastomosing the first cavity and the second cavity after cleaning the second cavity.

In any of the embodiments, the method for cleaning a cavity may include advancing the needle through multiple tissues to reach the second cavity.

In any of the embodiments, the method for cleaning a cavity may include advancing the needle through the endoscopic port at approximately 90 degrees to the tissue wall.

In any of the embodiments, the method for cleaning a cavity may include guiding the needle with fluoroscopic guidance.

In any of the embodiments, the method for cleaning a cavity may include using at least one of ultrasound computed tomography or magnetic resonance imaging guidance.

1. An anastomosis device comprising:
a base member defining a passageway between a first end and a second end of the base member,
wherein the base member has a cutting element to cut tissue from the first end;
a first support member connected to the base member proximal to the first end; and
a second support member connected to the base member proximal to the second end, wherein at least one of the first support member and the second support member is movable along a length of the base member to secure a portion of tissue between the first and second support members.

2. The anastomosis device of claim 1, wherein the base member is releasably engagable to a distal end of a delivery device.

3. The anastomosis device of claim 2, wherein the base member is configured to slide over the distal end of the delivery device.

4. The anastomosis device of claim 3, further comprising a supporting balloon that is inflatable and deflatable at the distal end of the delivery device, wherein the supporting balloon prevents the base member from detaching from the delivery device when inflated.

5. (canceled)

6. (canceled)

7. The anastomosis device of claim 2, wherein the delivery device includes a separate channel to house an electrical line that is configured to carry a current.

8. The anastomosis system of claim 2, wherein current is transmitted through a conductive delivery device contact on the delivery device to a conductive anastomosis contact on the second end of the base member when the conductive delivery device contact and the conductive anastomosis contact are in direct contact.

9. The anastomosis system of claim 2, further comprising a needle connected to the delivery device and configured to be extended forwardly from the base member.

10. The anastomosis device of claim 1, further comprising a sheath with an electrical line extending along the sheath and configured to transmit power from a proximal end of the delivery device to the distal end of the delivery device, wherein the sheath is configured to slide over the delivery device to contact the second end of the base member.

11. The anastomosis device of claim 1, wherein the first support member and the second support member are configured to clamp together tissue from at least two organs to anastomose the at least two organs.

12. The anastomosis device of claim 11, wherein the two organs are a duodenum and a gallbladder.

13. (canceled)

14. (canceled)

15. The anastomosis device of claim 1, wherein a shell of the base member provides insulation around the cutting element, wherein the cutting element is exposed through the shell along the first end of the base member.

16. The anastomosis device of claim 1, wherein the cutting element utilizes at least one of cauterization, perforation, monopolar or bipolar electrocautery, ultrasound cauterization, radio frequency cutting, mechanical cutting, laser cutting, or harmonic cutting.

17. The anastomosis device of claim 1, wherein the first support member and the second support member are configured to clamp a tissue wall of a gallbladder and a tissue wall of a duodenum therebetween.

18. The anastomosis device of claim 1, wherein the first support member is deformable.

19. (canceled)

20. (canceled)

21. The anastomosis device of claim 1, wherein the base member includes an engagement structure along at least a portion of an outside perimeter of a shell of the base member, wherein at least one of the first support member or the second support member is movable along the engagement structure.

22. The anastomosis device of claim 21, wherein the engagement structure is a series of sequential steps angled toward a center of the base member to allow at least one of the first support member or the second support member to move in one direction along the outside perimeter of the shell.

23. The anastomosis device of claim 22, wherein an inner circumference of one of the first support member or the second support member includes at least one wedge that is complementary to the steps.

24. The anastomosis device of claim 1, further comprising a pneumatic deployer configured to push one of the first support member and the second support member along the base member when inflated.

25. The anastomosis device of claim 1, further comprising a one-way valve attached to the base member.

26. (canceled)

27. An anastomosis method, comprising:
advancing an anastomosis device into a first cavity with a first tissue wall, the anastomosis device comprising a base member defining an inner passage between a first end and a second end, and a first support member and a second support member extending outwardly from the base member;
cutting the first tissue wall with the first end of the anastomosis device;
advancing the first support member through the first tissue wall to a second tissue wall of a second cavity;
cutting the second tissue wall with the first end of the anastomosis device;
advancing the first support member through the second tissue wall; and
clamping the first and second tissue walls between the first support member and the second support member.

28.-72. (canceled)
